(19) United States
(12) Patent Application Publication
(10) Pub. No.: US 2016/0361257 A1
(43) Pub. Date: Dec. 15, 2016

(54) COMPOSITION FOR INCREASING TESTOSTERONE LEVELS AND ENHANCING LIBIDO

(71) Applicant: Michael Farber, Livingston, NJ (US)

(72) Inventor: Michael Farber, Livingston, NJ (US)

(21) Appl. No.: 14/735,200

(22) Filed: Jun. 10, 2015

(51) Int. Cl. A61K 9/127; A23L 1/236; A61K 9/00; A23L 1/22; A61K 31/341; A61K 31/352

(52) U.S. Cl. CPC A61K 9/127; A61K 31/341; A61K 31/352; A61K 9/006; A23L 1/22; A23L 1/236; A23V 2002/00

(57) ABSTRACT

The present invention features a compressible delivery formulation for buccal delivery, dosage forms and methods of using the same for increasing testosterone levels in a subject. The formulation, dosage forms and methods comprise effective amounts of 3,4-divanillyltetrahydrofuran, 7-methoxyflavone, 7,8-benzoflavone.

### BACKGROUND

The subject matter disclosed generally relates to compositions and methods for increasing testosterone levels and enhancing libido, and more particularly to compositions and methods for releasing protein-bound testosterone, and preventing its degradation in vivo.

### RELATED PRIOR ART

Testosterone is the principal male androgen and is responsible for the development and maintenance of male sexual characteristics, including external virilization, sexual maturity at puberty, spermatogenesis, sexual behaviour, libido and erectile functioning. It also supports bone and muscle tissue development during growth. However, after physical maturity, the level of circulating testosterone starts to decline, possibly leading to a diminution in muscle mass. Therefore, there is a growing need for the development of some form of androgen replacement for the elimination of the effects encountered on muscle mass, which is a specific concern for men evolving in the sports and bodybuilding domains.

Several methods for re-establishing androgen levels to their pre-adult concentrations in men were developed with injectable preparations. U.S. Pat. No. 6,989,378, among others, relates to a novel androgen, (7α, 17β)-7-methyl-17-[(1-ox-oundecyl)oxy]estr-4en-3one (MENT undecanoate), having a good solubility in oily media and being particularly suitable for administration by means of injection. Injectable medias are normally fashioned in order to allow slow and sustained hormone release in the blood of the patient over various preset periods of time. However, the main problem with such inventions is that they usually end up providing inconsistent dosing because of a great variance in hormone release between the site of injection and the rest of the body. Moreover, injection of testosterone preparations usually entails very high concentrations from the moment of the administration followed by a period of subnormal hormone concentrations. Because of such uncontrolled release of the active agent, various side-effects developed in periods of high hormone concentrations, such as gynecomastia, water retention, edema and increased fat deposition.

Some methods of treatment for restoring the testosterone concentrations in adult males with declining levels focused on the administration of a metabolic precursor of testosterone. U.S. Pat. No. 5,880,117 relates to a method of administering the testosterone precursor 4-androstene-3β, 17β-diol as a means of increasing testosterone levels in humans. This invention proposes a compound which concentration peaks within 90 minutes of its administration and declines thereafter over a period of 3 to 4 hours. Even if the androgen preparation has shown easy conversion to testosterone in the physiologic environment, it still lacks constant reparation in the organs of predilection and can possibly entail various side-effects.

U.S. Pat. No. 6,451,782 is based on the administration of 4-androstene-3α, 17β-diol, a direct precursor hormone to testosterone, in order to increase testosterone levels in male subjects. However, even if conversion to testosterone has been demonstrated as being much more complete than in other cases, the release kinetic of the compound were still not ideal.

Other proposed methods of treating the present condition were related to the physiologic administration of synthetic androgen derivatives of testosterone such as methyltestosterone, fluoxymesterone and stanozol. Those compounds were alkylated at the 17^{th} carbon in order to restrain any reduction of the metabolite to its inactive from. Such innovation induced an increase in the bioavailability of the compound, which allowed a more constant release of the active agent in the physiologic environment. However, patients encountered possible risks of developing complications with liver functions, which highly diminishes the convenience of using such technology.

Steroidal based aromatase inhibitors, androsta-1,4,6-triene-3, 17-dione (ATD) specifically, have been cited in the literature on numerous occasions over the past thirty years. It was first used to elucidate the nature of the enzyme aromatizing androstenedione and testosterone. The effects of aromatase inhibition upon sex steroids in men (in this case older eugonadal men) were definitively and quantitatively studied wherein it was shown that administration of an aromatase inhibitor to 15 men over 65 caused significant decreases in estrogen and its related compounds and significantly increased testosterone.

It is well known that various flavones and flavanones can inhibit aromatase activity, such as chrysin and quercetin among others. The biggest problem with the administration of flavones and flavanones is their poor absorption characteristics due to enzymatic degradation in the intestine and poor absorption across the intestinal epithelium.

It would therefore be highly desirable to be provided with a potent fast acting aromatase inhibitor displaying only slight or negligible binding to the peripheral androgen receptors that would rapidly raise testosterone levels in male subjects, have a half-life allowing for at most twice daily ingestion and have sufficient binding to the hypothalamic androgen receptor sites to downregulate the feedback loop.

### SUMMARY OF THE INVENTION

According to an embodiment, there is provided a compressible delivery formulation for buccal delivery comprising:

a micronized powder base;

an effective amount of 3,4-divanillyltetrahydrofuran;

an effective amount of 7-methoxyflavone, 7,8-benzoflavone or a combination thereof; and

dessicated liposome formulation comprising at least one liposome containing at least one mucosal absorption enhancer.

The 3,4-divanillyltetrahydrofuran, 7-methoxyflavone and 7,8-benzoflavone may be micronized particles, nanonized particles, desiccated liposomal particles, or combinations thereof.

The 3,4-divanillyltetrahydrofuran, 7-methoxyflavone and 7,8-benzoflavone may be desiccated liposomal particles.

The mucosal absorption enhancer may be chosen from 23-lauryl ether, aprotinin, azone, benzalkonium chloride, cetylpyridinium chloride, cetyltrimethylammonium bromide, cyclodextrin, dextran sulfate, lauric acid, lauric acid/Propylene glycol, lysophosphatidylcholine, menthol, methoxysalicylate, methyloleate, oleic acid, piperine, methyl piperate, a piperine derivative, a methyl piperate derivative, phosphatidylcholine, polyoxyethylene, polysorbate 80, sodium EDTA, sodium glycocholate, sodium glycodeoxycholate, sodium lauryl sulfate, sodium salicylate, sodium taurocholate, sodium taurodeoxycholate, sodium deoxycholate, a sulfoxide, bile salts, an alkyl glycoside, and combinations thereof.

The mucosal absorption enhancer may be piperine.

The 3,4-divanillyltetrahydrofuran may be from an extract of nettle root, an isolated 3,4-divanillyltetrahydrofuran compound, or combinations thereof.

The micronized powder base may be chosen from an inert powdered base, an active powdered base having improved transmucosal permeation, or combinations thereof.

The inert powdered base may be chosen from a maltodextrin, a microcrystalline cellulose, sucrose, xylitol, sorbitol, mannitol, or combinations thereof.

The active powdered base may be chosen from a caffeine, theobromine, theophylline, a plant extract with bioavailable components, and creatine.

The inert powdered base may be a maltodextrin.

The formulation may be further comprising a flavoring agent.

The flavoring agent may be chosen from orange flavor, lemon flavor, grapefruit flavor, blueberry flavor, raspberry flavor, strawberry flavor, peach flavor, grape flavor, apple flavor, mango flavor, banana flavor, mint flavor, cinnamon flavor, vanilla flavor, butterscotch flavor, caramel flavor chocolate flavor, and combinations thereof.

The mint flavor may be chosen from spearmint flavor and peppermint flavor, and combinations thereof.

The formulation may be further comprising a sweetener.

The sweetener may be chosen from glucose, fructose, aspartame, cyclamate, saccharin, stevia, sucralose, brazzein, curculin, erythritol, glycyrrhizin, glycerol, hydrogenated starch hydrolysates, inulin, isomalt, lactitol, Luo han guo, mabinlin, maltitol, malto-oligosaccharide, mannitol, miraculin, monatin, monellin, osladin, pentadin, sorbitol, tagatose, thaumatin, xylitol, acesulfame potassium, alitame, salt of aspartame-acesulfame, dulcin, glucin, neohesperidin dihydrochalcone, neotame and combinations thereof.

According to another embodiment, there is provided a dosage form comprising the formulation of the present invention in combination with a pharmaceutically acceptable carrier.

The 3,4-divanillyltetrahydrofuran, 7-methoxyflavone and 7,8-benzoflavone may be from about 5 mg to about 50 mg per dosage form.

The 3,4-divanillyltetrahydrofuran, 7-methoxyflavone and 7,8-benzoflavone may be from about 20 mg to about 30 mg per dosage form.

The absorption enhancer may be piperine, and the piperine is from about 400 μg to about 800 μg per dosage form.

The dosage form may be chosen from a chewable tablet, a fast disintegrating tablet, a chewing gum, and a granule.

The dosage form may be chosen from a jelly, a gel, a film, a lozenge, a toothpaste, an ointment, a liquid and a spray.

The pharmaceutically acceptable carrier may be a hydrocolloid.

The hydrocolloid may be chosen from agar, agarose, alginates, carrageenan (iota, kappa, lambda), cellulosics, chitosan, gelatin, gellan gum, guar gum, gum arabic, locust bean gum, pectin, soybean gel, starch, whey protein, xanthan gum, chewing gum, a base gum and derivatives thereof and combinations thereof.

The buccal delivery may be mouth transmucosal delivery, sublingual delivery, or a combination thereof.

According to another embodiment, there is provided a method of increasing testosterone levels and enhancing libido comprising:

(a) administering to a subject in need thereof a dosage form as claimed in claims 16-23 to increase testosterone levels and/or enhance libido.

The administering may be twice daily.

The administering twice daily may be at 7 AM and 7 PM.

According to another embodiment, there is provided a use of a formulation of the present invention, or a dosage form of the present invention for the preparation of a medicament for increasing testosterone levels and enhancing libido.

According to another embodiment, there is provided a use of a formulation of the present invention, or a dosage form of the present invention for increasing testosterone levels and enhancing libido.

The following terms are defined below.

The term “Therapeutically effective amount” or “effective amount” is intended to mean the amount of the compound and/or composition that is effective to achieve its intended purpose.

“Transdermally absorbed” is intended to mean the delivery of a compound by passage through the skin and into the blood stream.

“Transmucosal” is intended to mean the delivery of a compound by passage of the compound through the mucosal tissue and into the blood stream.

“Carriers” or “vehicles” are intended to mean carrier materials suitable for compound administration and include any such material known in the art such as, for example, any liquid, lotion, gel, solvent, liquid diluent, solubilizer, or the like, which is non-toxic and which does not interact with any components of the composition in a deleterious manner.

The term “micronized” or “micronization” are intended to mean the process of reducing the average diameter of a solid material's particles to produce particles that are only a few micrometers in diameter.

The term “liposome” is intended to mean an artificially-prepared vesicle composed of a lipid bilayer. The liposome can be used as a vehicle for administration of nutrients and pharmaceutical drugs, and it can be prepared by disrupting biological membranes (such as by sonication). Liposomes are composed of natural phospholipids, and may also contain mixed lipid chains with surfactant properties (e.g., egg phosphatidylethanolamine). The major types of liposomes are the multilamellar vesicle (MLV), the small unilamellar vesicle (SUV), and the large unilamellar vesicle (LUV).

The term “pharmaceutical acceptable carrier” is intended to mean a preservative solution, a saline solution, an isotonic (about 0.9%) saline solution, or about a 5% albumin solution, suspension, sterile water, phosphate buffered saline, and the like. Other buffering agents, dispersing agents, and inert non-toxic substances suitable for delivery to a patient may be included in the compositions of the present invention. The compositions may be solutions, suspensions or any appropriate formulation suitable for administration, and are typically sterile and free of undesirable particulate matter. The compositions may be sterilized by conventional sterilization techniques.

The term “inert” is intended to mean that the powdered base does not illicit any therapeutic physiological effect in organism consuming the formulation.

The term “active” is intended to mean that the powdered base does have a therapeutic physiological effect in organism consuming the formulation.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### DETAILED DESCRIPTION

In embodiment there is disclosed a compressible delivery formulation for buccal delivery. The formulation comprises

a micronized powder base,

an effective amount of 3,4-divanillyltetrahydrofuran,

an effective amount of 7-methoxyflavone, 7,8-benzoflavone or a combination thereof,

and a desiccated liposome formulation comprising at least one liposome containing at least one mucosal absorption enhancer.

In second embodiment, there is disclosed a dosage form comprising the formulation of the present invention in combination with a pharmaceutically acceptable carrier.

In third embodiment, there is disclosed a method of increasing testosterone levels and enhancing libido by administering to a subject in need thereof a dosage form of the present invention to increase testosterone levels and/or enhance libido.

According to the first embodiment there is disclosed a compressible delivery formulation for buccal delivery. The formulation comprises a micronized powder base, an effective amount of 3,4-divanillyltetrahydrofuran, an effective amount of 7-methoxyflavone, 7,8-benzoflavone, or a combination thereof, and a desiccated liposome formulation comprising at least one liposome containing at least one mucosal absorption enhancer.

As used herein, the term buccal delivery is intended to mean “in the mouth” and include transmucosal delivery, sublingual delivery, or a combination of both.

### Micronized Powder Base

According to an embodiment, the micronized powder base may be an inert powdered base, an active powdered base having improved transmucosal permeation, or combinations thereof.

Examples of inert powdered base include without limitations maltodextrins, microcrystalline cellulose, sucrose, xylitol, sorbitol, and mannitol. As used herein, the term inert is intended to mean that the powdered base does not illicit any therapeutic physiological effect in organism consuming the formulation.

Maltodextrins are polysaccharides that are used as a food additive. They are produced from starch by partial hydrolysis and are usually found as a white hygroscopic spraydried powders. Maltodextrins are easily digestible, being absorbed as rapidly as glucose, and might be either moderately sweet or almost flavorless. They are commonly used for the production of sodas and candy and can also be found as an ingredient in a variety of other processed foods.

Microcrystalline cellulose is a term for refined wood pulp and is used as a texturizer, an anti-caking agent, a fat substitute, an emulsifier, an extender, and a bulking agent in food production. The most common form is used in vitamin supplements or tablets.

Sucrose is the organic compound commonly known as table sugar and sometimes called saccharose. A white, odorless, crystalline powder with a sweet taste, it is best known for its nutritional role.

Xylitol, sorbitol, and mannitol are sugar alcohol sweetener used as a naturally occurring sugar substitute.

As used herein, the term “active” is intended to mean that the powdered base does have a therapeutic physiological effect in organism consuming the formulation.

Non limiting examples of active powdered base are caffeine, theobromine, theophylline, a plant extract with bioavailable components, and creatine. Examples of plant extract with bioavailable components include green coffee extract, guarana extracts, Yerba mate extracts, tea extract, citrus aurantium extracts, and combinations thereof. Preferred active powdered base are include caffeine, theobromine, and creatine. Preferably, the caffeine is a salt of caffeine, such as for example dicaffeine malate, caffeine citrate, caffeine hydrochloride, or combinations thereof.

### Micronization, Nanonization, Liposomal Formulations

According to another embodiment of the present invention, the 3,4-divanillyltetrahydrofuran, 7-methoxyflavone and 7,8-benzoflavone may be formulated as micronized or nanonized particles. Also, they may be formulated as dessicated liposomomal formulations.

### Liposome Formulation

According to another embodiment, the formulation of the present invention may comprise dessicated liposome formulation that comprising at least one liposome containing at least one of 3,4-divanillyltetrahydrofuran, 7-methoxyflavone and 7,8-benzoflavone. The liposome formulation may contain more than one type of liposome, each of which may be loaded with one or more of 3,4-divanillyltetrahydrofuran, 7-methoxyflavone and 7,8-benzoflavone. According to an embodiment, the liposome(s) are loaded with the compound(s) (3,4-divanillyltetrahydrofuran, 7-methoxyflavone and 7,8-benzoflavone) with sufficient amounts to achieve unitary doses (dosage forms) as defined below. Typically, such desiccated liposome have been found capable of delivering from about 10 μg to about 500 mg of the 3,4-divanillyltetrahydrofuran, 7-methoxyflavone or 7,8-benzoflavone, or from about 100 μg to about 500 mg, or from about 200 μg to about 500 mg, or from about 300 μg to about 500 mg, or from about 400 μmg to about 500 mg, or from about 500 μg to about 500 mg, or from about 600 μg to about 500 mg, or from about 700 μg to about 500 mg, or from about 800 μg to about 500 mg, or from about 900 μg to about 500 mg, or from about 1 mg to about 500 mg, or from about 10 mg to about 500 mg, or from about 10 mg to about 400 mg, or from about 10 mg to about 300 mg, or from about 10 mg to about 200 mg, or from about 10 mg to about 100 mg, or from about 10 mg to about 50 mg, or from about 10 mg to about 25 mg, or from about 25 mg to about 500 mg, or from about 25 mg to about 400 mg, or from about 25 mg to about 300 mg, or from about 25 mg to about 200 mg, or from about 25 mg to about 100 mg, or from about 25 mg to about 50 mg, or from about 50 mg to about 500 mg, or from about 50 mg to about 400 mg, or from about 50 mg to about 300 mg, or from about 50 mg to about 200 mg, or from about 50 mg to about 100 mg, or from about 100 mg to about 500 mg, or from about 100 mg to about 400 mg, or from about 100 mg to about 300 mg, or from about 100 mg to about 200 mg, or from about 200 mg to about 500 mg, or from about 200 mg to about 400 mg, or from about 200 mg to about 300 mg, or from about 300 mg to about 500 mg, or from about 300 mg to about 400 mg, or from about 400 mg to about 500 mg. Without wishing to be bound by theory, the amount of compound will vary according to the 3,4-divanillyltetrahydrofuran, 7-methoxyflavone and 7,8-benzoflavone selected, and the desired dosage required to obtain the desired health beneficial effect or therapeutic effect. The person skilled in the art is capable of determining the amount of compound necessary to be loaded into the liposomes in order to achieve the desired amounts in the final dosage forms.

The use of desiccated liposomes in the formulation of the present invention allows for the preparation of formulations having low water content that have long shelf life. Furthermore, upon contact with the bodily fluids in the body cavity in proximity to the target mucosa (e.g. buccal, vaginal or anal), the desiccated liposomes present in the formulation of the present invention rehydrate and the liposomes are shown under microscope to be normal sphericals (i.e filled with water) to provide one or more Iiposome encapsulated compounds in situ. Without wishing to be bound by theory, it is believed that upon contact with the mucosa, the content of the liposome is delivered transmucosally directly into the systemic circulation. Furthermore, the use of desiccated liposomes also contributes to masking of the taste of some of the compounds that they contain, thereby helping to provide a more palatable delivery system.

According to an embodiment, unilamellar liposomes may be used to contain water soluble compounds for higher bioavailability and faster absorption.

### 3,4-Divanillyltetrahydrofuran

3,4-Divanillyltetrahydrofuran is a lignan found in a Urtica dioica (stinging nettle) subspecies. This same compound may also be found in other lignan-containing plant sources such as Linum usitatissimum (flax seed).

The compound has been found to occupy binding sites of sex hormone-binding globulin (SHBG), thereby reducing the ability of SHBG to bind additional steroid hormones such as estrogens and androgens, mostly testosterone and estradiol. Certain extracts of stinging nettle are therefore used by some bodybuilders in an effort to increase free testosterone. According to an embodiment, the 3,4-Divanillyltetrahydrofuran may therefore be from an extract of nettle root containing at least 80% 3,4-Divanillyltetrahydrofuran, standardized. Preferably, the nettle root extract contains at least 98% 3,4-Divanillyltetrahydrofuran, standardized.

### 7-Methoxyflavone

7-methoxyflavone is an aromatase inhibitor. Aromatase inhibitors work by inhibiting the action of the enzyme aromatase, which converts androgens into estrogens by a process called aromatization. 7-methoxyflavone is a compound having poor bioavailability of about 5% when ingested. According to an embodiment of the present invention, from about 5 mg to 50 mg, and preferably from about 20 mg to 30 mg of 7-methoxyflavone may be included in the formulations and dosage forms of the present invention, when delivered sublingually or transmucosally. According to an embodiment, the half-life of 7-methoxyflavone is about 8-12 h when delivered sublingually or transmucosally. According to an embodiment, the 7-methoxyflavone may be from a plant extract. 7-methoxyflavone has been found in Meliaceae and Rutaceae plants. According to an embodiment, the 7-methoxyflavone may therefore be from an extract containing at least 90% 7-methoxyflavone, standardized. Preferably, extract contains at least 100% 7-methoxyflavone, standardized.

### 7,8-Benzoflavone

7,8-benzoflavone also known as alpha-Naphthoflavone, and 2-phenyl-benzo(h)chromen-4-one, is a synthetic flavone derivative. It can be prepared from 2-naphthol and cinnamaldehyde. 7,8-benzoflavone is a potent inhibitor of the enzyme aromatase, the enzyme that converts testosterone to estrogen.

According to an embodiment of the present invention, from about 5 mg to 50 mg, and preferably from about 20 mg to 30 mg of 7,8-benzoflavone may be included in the formulations and dosage forms of the present invention, when delivered sublingually or transmucosally. According to an embodiment, the half-life of 7,8-benzoflavone is about 1-2h when delivered sublingually or transmucosally.

### Mucosal Absorption Enhancers

According to another embodiment, the formulation of the present invention may comprise desiccated liposome formulation that comprises at least one liposome containing at least one mucosal absorption enhancer. The liposome formulation may contain more than one type of liposome, each of which may be loaded with one or more different mucosal absorption enhancer. According to an embodiment, the liposome(s) are loaded with the mucosal absorption enhancer(s) with sufficient amounts of mucosal absorption enhancer to achieve unitary doses (dosage forms) capable of delivering from about 10 μg to about 500 mg of the mucosal absorption enhancer(s), or from about 100 μg to about 500 mg, or from about 200 μg to about 500 mg, or from about 300 μg to about 500 mg, or from about 400 μg to about 500 mg, or from about 500 μg to about 500 mg, or from about 600 μg to about 500 mg, or from about 700 μg to about 500 mg, or from about 800 μg to about 500 mg, or from about 900 μg to about 500 mg, or from about 1 mg to about 500 mg, or from about 10 mg to about 500 mg, or from about 10 mg to about 400 mg, or from about 10 mg to about 300 mg, or from about 10 mg to about 200 mg, or from about 10 mg to about 100 mg, or from about 10 mg to about 50 mg, or from about 10 mg to about 25 mg, or from about 25 mg to about 500 mg, or from about 25 mg to about 400 mg, or from about 25 mg to about 300 mg, or from about 25 mg to about 200 mg, or from about 25 mg to about 100 mg, or from about 25 mg to about 50 mg, or from about 50 mg to about 500 mg, or from about 50 mg to about 400 mg, or from about 50 mg to about 300 mg, or from about 50 mg to about 200 mg, or from about 50 mg to about 100 mg, or from about 100 mg to about 500 mg, or from about 100 mg to about 400 mg, or from about 100 mg to about 300 mg, or from about 100 mg to about 200 mg, or from about 200 mg to about 500 mg, or from about 200 mg to about 400 mg, or from about 200 mg to about 300 mg, or from about 300 mg to about 500 mg, or from about 300 mg to about 400 mg, or from about 400 mg to about 500 mg. Without wishing to be bound by theory, the amount of mucosal absorption enhancer will vary according to the mucosal absorption enhancer selected, and the desired dosage required to obtain the desired health beneficial effect or therapeutic effect. The person skilled in the art is capable of determining the amount of mucosal absorption enhancer necessary to be loaded into the liposomes in order to achieve the desired amounts in the final dosage forms.

The use of desiccated liposomes in the formulation of the present invention allows for the preparation of formulations having low water content that have long shelf life. Furthermore, upon contact with the bodily fluids in the body cavity in proximity to the target mucosa (e.g. buccal, vaginal or anal), the desiccated liposomes present in the formulation of the present invention rehydrate and the liposomes are shown under microscope to be normal sphericals (i.e filled with water) to provide one or more liposome encapsulated mucosal absorption enhancer in situ. Without wishing to be bound by theory, it is believed that upon contact with the mucosa (or the saliva of the mouth), the content of the liposome is delivered transmucosally directly into the systemic circulation. Furthermore, the use of desiccated liposomes also contributes to masking of the taste of some of the mucosal absorption enhancer that they contain, thereby helping to provide a more palatable delivery system.

According to an embodiment, unilamellar liposomes may be used to contain water soluble mucosal absorption enhancer for higher bioavailability and faster absorption.

According to another embodiment, the formulation of the present invention may further comprise a mucosal absorption enhancer for improved transmucosal permeation. According to an embodiment, the mucosal absorption enhancer may be one of the compounds included in one of the liposome formulation used in the present invention. According to another embodiment, the mucosal absorption enhancer may be added as a further ingredient of the formulation of the present invention, without the benefit of liposome encapsulation.

Examples of mucosal absorption enhancer include but are not limited to 23-lauryl ether, aprotinin, azone, benzalkonium chloride, cetylpyridinium chloride, cetyltrimethylammonium bromide, cyclodextrin, dextran sulfate, lauric acid, lauric acid/Propylene glycol, lysophosphatidylcholine, menthol, methoxysalicylate, methyloleate, oleic acid, piperine, methyl piperate, a piperine derivative, a methyl piperate derivative, phosphatidylcholine, polyoxyethylene, polysorbate 80, sodium EDTA, sodium glycocholate, sodium glycodeoxycholate, sodium lauryl sulfate, sodium salicylate, sodium taurocholate, sodium taurodeoxycholate, sodium deoxycholate, a sulfoxide, bile salts, and an alkyl glycoside. Preferably, the mucosal absorption enhancer is piperine. Most preferably, the mucosal absorption enhancer is liposome encapsulated piperine. Most preferable, liposome encapsulated piperine is used in about 400 μg to about 800 μg per dosage form.

### Flavoring and Sweetening Agent

According to another embodiment, the formulation of the present invention may further comprise flavoring and sweetening agents. The flavoring and sweetening agents further improve the palatability and mouth feel of the formulation of the present invention.

For example, the flavoring agent may be chosen from orange flavor, lemon flavor, grapefruit flavor, blueberry flavor, raspberry flavor, strawberry flavor, peach flavor, grape flavor, apple flavor, mango flavor, banana flavor, mint flavor, cinnamon flavor, vanilla flavor, butterscotch flavor, caramel flavor chocolate flavor, and combinations thereof.

Preferably, the flavoring may be a mint flavor. Most preferably, the flavoring may be chosen from spearmint flavor and peppermint flavor, and combinations thereof.

Examples of sweeteners include but are not limited to glucose, fructose, aspartame, cyclamate, saccharin, stevia, sucralose, brazzein, curculin, erythritol, glycyrrhizin, glycerol, hydrogenated starch hydrolysates, inulin, isomalt, lactitol, Luo han guo, mabinlin, maltitol, malto-oligosaccharide, mannitol, miraculin, monatin, monellin, osladin, pentadin, sorbitol, tagatose, thaumatin, xylitol, acesulfame potassium, alitame, salt of aspartame-acesulfame, dulcin, glucin, neohesperidin dihydrochalcone, neotame and combinations thereof.

### Dosage Forms

According to the second embodiment, the formulations of the present inventions may be included into dosage forms, along with suitable pharmaceutically acceptable carriers, and the dosage form contains from about 10 μg to about 500 mg of the ingredients.

Examples of suitable dosage forms for transmucosal delivery include but are not limited to chewable tablets, fast disintegrating tablets, jellies, gels, chewing gum, granules, films, lozenges, toothpastes and liquids (e.g. mouthwashes), which may be administered buccally (i.e. through the buccal mucosa), as well as ointments, and suppositories, which may be administered through the vaginal and anal mucosa.

Preferred dosage forms are chosen from chewable tablets, fast disintegrating tablets, chewing gums, granules, and suppositories.

Other suitable dosage forms include a jelly, a gel, a film, a lozenge, a toothpaste, an ointment, a liquid and a spray.

### Pharmaceutically Acceptable Carriers

According to an embodiment, the dosage form of the present invention may contain any suitable pharmaceutically acceptable carriers as known in the art.

According to an embodiment, a preferred pharmaceutically acceptable carrier is a hydrocolloid. As used herein, hydrocolloids are substances that form a gel in the presence of water. Examples of hydrocolloids include but are not limited to agar, agarose, alginates, carrageenan (iota, kappa, lambda), cellulosics, chitosan, gelatin, gellan gum, guar gum, gum arabic, locust bean gum, pectin, soybean gel, starch, whey protein, xanthan gum, chewing gum, a base gum and derivatives thereof and combinations thereof.

### Preparation of Formulations and of Dosage Forms

According to another embodiment, there is disclosed a process for the preparation of a formulation for buccal delivery by spraying a liposome formulation suspended in a water based solvent on a micronized powder base (as descrived above). The liposome formulation comprises at least one liposome containing at least one mucosal absorption enhancer (as descrived above). Spraying is effected preferably in a fluidized bed, or any suitable apparatus. Spraying is effected at a temperature at a nozzle head aperture of about 60° C. or less. Spraying of the liposome formulation in this fashion causes the water based solvent to evaporate, as well as dissipate the heat present in the water based solvent, and results in desiccation of the liposome without damaging them. Similar processes may be employed for the preparation of desiccated liposomes for the 3,4-divanillyltetrahydrofuran, 7-methoxyflavone and 7,8-benzoflavone.

According to an embodiment, about 0.4 L to about 1.0 L of the liposome formulation suspended in a water based solvent is sprayed for each 1 kg of the micronized powder base. Therefore, the volume of liposome formulation suspended in a water based solvent sprayed for each 1 kg of the micronized powder base may be from about 0.4 L to about 0.5 L, or from about 0.4 L to about 0.6 L, or from about 0.4 L to about 0.7 L, or from about 0.4 L to about 0.8 L, or from about 0.4 L to about 0.9 L, or from about 0.4 L to about 1.0 L, or from about 0.5 L to about 0.6 L, or from about 0.5 L to about 0.7 L, or from about 0.5 L to about 0.8 L, or from about 0.5 L to about 0.9 L, or from about 0.5 L to about 1.0 L, or from about 0.6 L to about 0.7 L, or from about 0.6 L to about 0.8 L, or from about 0.6 L to about 0.9 L, or from about 0.6 L to about 1.0 L, or from about 0.7 L to about 0.8 L, or from about 0.7 L to about 0.9 L, or from about 0.7 L to about 1.0 L, or from about 0.8 L to about 0.9 L, or from about 0.8 L to about 1.0 L, or from about 0.9 L to about 1.0 L. Preferably, the volume is from about 0.4 L to about 0.7 L for each 1 kg of the micronized powder base.

According to an embodiment, the liposome formulation comprises the ingredients (e.g. mucosal absorption enhancer, 3,4-divanillyltetrahydrofuran, 7-methoxyflavone and 7,8-benzoflavone) in an amount sufficient to form unitary dosage forms that contain from about 10 μg to about 500 mg of ingredients. Without wishing to be bound by theory, the amount of ingredients will vary according to the compound selected, and the desired dosage required to obtain the desired health beneficial effect or therapeutic effect. The person skilled in the art is capable of determining the amount of compound necessary to be loaded into the liposomes in order to achieve the desired amounts in the final dosage forms.

According to an embodiment, the water based solvent may be water, or a mixture of water and alcohol. For example, the alcohol may be ethanol. According to another embodiment, the water based solvent may further comprise flavoring agents, sweetening agent, or combinations thereof.

According to another embodiment, there is provided a process for the preparation of a solid dosage form by compressing a formulation of the present invention as described above, in combination with a pharmaceutically acceptable carrier as described above, without a heat treatment capable of causing degradation of the formulation. As used herein, without a heat treatment capable of causing degradation of the formulation is intended to mean that only compression is used during the preparation of the dosage form. No heat treatment that would damage the liposome present in the formulation of the present invention should be used. Liposomes are notoriously sensitive to increased temperatures that damage the phospholipids constituting their bilayers; and hence temperature should be kept as low as possible and not exceeding 60° C. when preparing the dosage form from the formulation of the present invention.

According to another embodiment, there is provided a method of increasing testosterone levels and enhancing libido by administering to a subject in need thereof a dosage form of the present invention to increase testosterone levels and enhance libido. Preferably, administering is performed twice daily, and most preferably twice daily at 7 AM and 7 PM.

In use, the formulation and dosage form of the present invention are believed to increase testosterone levels through the concerted action of 3,4-divanillyltetrahydrofuran which acts to free testosterone from the sex hormone-binding globulin (SHBG), and the aromatase inhibitors 7-methoxyflavone and 7,8-benzoflavone, which prevent the conversion of testosterone to estrogen through action of the aromatase enzyme.

The formulation and dosage forms of the present invention provide an efficient vehicle to administer 3,4-divanillyltetrahydrofuran, 7-methoxyflavone and 7,8-benzoflavone and increase their bioavailability to levels superior to those obtained with oral (i.e. ingestion) administration. Transmucosal and sublingual administration bypasses first pass metabolism and contributes to reaching the higher than normal bioavailability for those compounds.

The present invention will be more readily understood by referring to the following examples which are given to illustrate the invention rather than to limit its scope.

### EXAMPLE 1

### Preparation of a Formulation #1

Micronized maltodextrin (micronized powder base) is mixed with micronized powder of 3,4-divanillyltetrahydrofuran and 7-methoxyflavone and 7,8-benzoflavone, which are mixed with desiccated liposomes containing piperine (as a mucosal absorption enhancer). Sweetener and flavoring is added, and the powder is mixed in a fluid bed.

| Ingredient | Amount (g) |
|---|---|
| micronized maltodextrin | 0.060 |
| micronized powder of 3,4- | 0.040 |
| divanillyltetrahydrofuran | |
| micronized 7-methoxyflavone | 0.040 |
| micronized 7,8-benzoflavone | 0.040 |
| desiccated liposomes containing piperine | 0.0008 |
| sweetener | 0.003 |
| flavoring | 0.004 |

### EXAMPLE 2

### Preparation of a Formulation #2

Micronized maltodextrin (micronized powder base) is mixed with desiccated liposomes containing valine ethyl ester nitrate [VEEN—a compound of formula (I)] and desiccated liposomes containing puerarin extract from pueraria lobata (containing 98% puerarin) are mixed with desiccated liposomes containing piperine (as a mucosal absorption enhancer). Sweetener and flavoring is added, and the powder is mixed in a fluid bed.

| Ingredient | Amount (g) |
|---|---|
| micronized maltodextrin | 0.080 |
| desiccated liposomes of 3,4- | 0.025 |
| divanillyltetrahydrofuran | |
| desiccated liposomes of 7- | 0.020 |
| methoxyflavone | |
| desiccated liposomes of 7,8- | 0.020 |
| benzoflavone | |
| desiccated liposomes containing piperine | 0.0008 |
| sweetener | 0.003 |
| flavoring | 0.004 |

Solutions containing the liposomes are sprayed onto the micronized maltodextrin base in a fluid bed. Spraying is effected at a temperature at a nozzle head aperture of about 60° C., and sweetener and flavoring are added.

### EXAMPLE 3

### Compressed Dosage Forms

The compressible powder formulation combined with excipients of examples 1 and 2 are compressed into dosage form, which contain individual ingredients in the following amounts:

| | |
|---|---|
| | Amount |
| Ingredient | mg/dosage |

Dosage form #1

| | |
|---|---|
| micronized maltodextrin | 60 |
| micronized powder of 3,4- | 40 |
| divanillyltetrahydrofuran | |
| micronized 7-methoxyflavone | 40 |
| micronized 7,8-benzoflavone | 40 |
| desiccated liposomes | 8 |
| containing piperine | |
| sweetener | 3 |
| flavoring | 4 |
| Microcrystalline cellose | 90 |
| Magnesium stearate | 9 |
| Silicon dioxide | 6 |

Dosage form #2

| | |
|---|---|
| micronized maltodextrin | 80 |
| desiccated liposomes | 25 |
| containing 3,4- | |
| divanillyltetrahydrofuran | |
| desiccated liposomes | 20 |
| containing 7-methoxyflavone | |
| desiccated liposomes | 20 |
| containing 7,8-benzoflavone | |
| desiccated liposomes | 8 |
| containing piperine | |
| sweetener | 3 |
| flavoring | 4 |
| Microcrystalline cellose | 73.85 |
| Magnesium stearate | 7.39 |
| Silicon dioxide | 4.92 |

While preferred embodiments have been described above and illustrated in the accompanying drawings, it will be evident to those skilled in the art that modifications may be made without departing from this disclosure. Such modifications are considered as possible variants comprised in the scope of the disclosure.

1. A compressible delivery formulation for buccal delivery comprising:
a micronized powder base;
an effective amount of 3,4-divanillyltetrahydrofuran;
an effective amount of 7-methoxyflavone, 7,8-benzoflavone or a combination thereof; and
dessicated liposome formulation comprising at least one liposome containing at least one mucosal absorption enhancer.

2. The formulation of claim 1, wherein said 3,4-divanillyltetrahydrofuran, said 7-methoxyflavone and said 7,8-benzoflavone are micronized particles, nanonized particles, desiccated liposomal particles, or combinations thereof.

3. The formulation of claim 2, wherein said 3,4-divanillyltetrahydrofuran, said 7-methoxyflavone and said 7,8-benzoflavone are desiccated liposomal particles.

4. The formulation of claim 1, wherein said mucosal absorption enhancer is chosen from 23-lauryl ether, aprotinin, azone, benzalkonium chloride, cetylpyridinium chloride, cetyltrimethylammonium bromide, cyclodextrin, dextran sulfate, lauric acid, lauric acid/Propylene glycol, lysophosphatidylcholine, menthol, methoxysalicylate, methyloleate, oleic acid, piperine, methyl piperate, a piperine derivative, a methyl piperate derivative, phosphatidylcholine, polyoxyethylene, polysorbate 80, sodium EDTA, sodium glycocholate, sodium glycodeoxycholate, sodium lauryl sulfate, sodium salicylate, sodium taurocholate, sodium taurodeoxycholate, sodium deoxycholate, a sulfoxide, bile salts, an alkyl glycoside, and combinations thereof.

5. The formulation of claim 4, wherein said mucosal absorption enhancer is piperine.

6. The formulation of claim 1, wherein said 3,4-divanillyltetrahydrofuran is from an extract of nettle root, an isolated 3,4-divanillyltetrahydrofuran compound, or combinations thereof.

7. The formulation of claim 1, wherein said micronized powder base is chosen from an inert powdered base, an active powdered base having improved transmucosal permeation, or combinations thereof.

8. The formulation of claim 7, wherein said inert powdered base is chosen from a maltodextrin, a microcrystalline cellulose, sucrose, xylitol, sorbitol, mannitol, or combinations thereof.

9. The formulation of claim 7, wherein said active powdered base is chosen from a caffeine, theobromine, theophylline, a plant extract with bioavailable components, and creatine.

10. The formulation of claim 8, wherein said inert powdered base is a maltodextrin.

11. The formulation of claim 1 further comprising a flavoring agent.

12. The formulation of claim 11, wherein said flavoring agent is selected from the group consisting of orange flavor, lemon flavor, grapefruit flavor, blueberry flavor, raspberry flavor, strawberry flavor, peach flavor, grape flavor, apple flavor, mango flavor, banana flavor, mint flavor, cinnamon flavor, vanilla flavor, butterscotch flavor, caramel flavor chocolate flavor, and combinations thereof.

13. The formulation of claim 12, wherein said mint flavor is selected from the group consisting of spearmint flavor and peppermint flavor, and combinations thereof.

14. The formulation of claim 1 further comprising a sweetener.

15. The formulation of claim 14, wherein said sweetener is selected from the group consisting of glucose, fructose, aspartame, cyclamate, saccharin, stevia, sucralose, brazzein, curculin, erythritol, glycyrrhizin, glycerol, hydrogenated starch hydrolysates, inulin, isomalt, lactitol, Luo han guo, mabinlin, maltitol, malto-oligosaccharide, mannitol, miraculin, monatin, monellin, osladin, pentadin, sorbitol, tagatose, thaumatin, xylitol, acesulfame potassium, alitame, salt of aspartame-acesulfame, dulcin, glucin, neohesperidin dihydrochalcone, neotame and combinations thereof.

16. A dosage form comprising the formulation of claim 1 in combination with a pharmaceutically acceptable carrier.

17. The dosage form of claim 16, wherein each of said 3,4-divanillyltetrahydrofuran, 7-methoxyflavone and 7,8-benzoflavone is from about 5 mg to about 50 mg per dosage form.

18. The dosage form of claim 16, wherein said wherein each of said 3,4-divanillyltetrahydrofuran, 7-methoxyflavone and 7,8-benzoflavone is from about 20 mg to about 30 mg per dosage form.

19. The dosage form of claim 16, wherein said absorption enhancer is piperine, and said piperine is from about 400 μg to about 800 μg per dosage form.

20. The dosage form of 16 wherein said dosage form is selected from the group consisting of a chewable tablet, a fast disintegrating tablet, a chewing gum, a granule, a jelly, a gel, a film, a lozenge, a toothpaste, an ointment, a liquid and a spray.

21. A method of increasing testosterone levels and enhancing libido comprising administering to a subject in need thereof a dosage form according to claim 16 to increase testosterone levels and/or enhance libido.
